(19) United States
(12) Patent Application Publication
(10) Pub. No.: US 2013/0090601 A1
(43) Pub. Date: Apr. 11, 2013

(54) FEEDING TUBE APERTURE

(71) Applicant: Henry Kiefer, Grand Junction, CO (US)

(72) Inventor: Henry Kiefer, Grand Junction, CO (US)

(21) Appl. No.: 13/253,945

(22) Filed: Oct. 5, 2011

(51) Int. Cl. A61M 39/00

(52) U.S. Cl. 604/175

(57) ABSTRACT

The present invention is a plastic feeding tube aperture utilized during a gavage process that includes a generally domed base that receives the feeding tube, a flange that is disposed below the generally domed base and a plurality of uniformly spaced feeding apertures that include 4 large apertures and one small aperture that are disposed on the generally domed base and extend through the feeding tube aperture. The feeding tube aperture also includes a generally cylindrical shaped plastic tube anchor that secures the feeding tube aperture in place within a feeding opening disposed on a user's body and one or more foam booties that are placed between the feeding tube aperture and the feeding opening to comfortably secure the feeding tube aperture within the feeding opening.

### TECHNICAL FIELD & BACKGROUND

Currently there are limited alternatives to equipment that offers medical patients a comfortable, easy-to-use opening attachment for feed tubes. Traditional apertures can puncture or rip the internal tissue lining of an individual potentially causing necrosis and possible infection.

The present invention generally relates to a feeding tube component. More specifically, the invention is a feeding tube aperture.

It is an object of the invention to provide a feeding tube aperture that facilitates the gavage process.

It is an object of the invention to provide a feeding tube aperture that holds a feeding tube in a perpendicular position in relation to a user's feeding opening without tearing into the user's surrounding body tissue.

It is an object of the invention to provide a feeding tube aperture that reduces contagions and allergic reactions during the gavage process.

What is really needed is a feeding tube aperture that facilitates the gavage process that holds a feeding tube in a perpendicular position in relation to a user's feeding opening without tearing into the user's surrounding body tissue that reduces contagions and allergic reactions during the gavage process.

### BRIEF DESCRIPTION OF THE DRAWINGS

The present invention will be described by way of exemplary embodiments, but not limitations, illustrated in the accompanying drawings in which like references denote similar elements, and in which:

FIG. 1A illustrates a front perspective view of a feeding tube aperture, in accordance with one embodiment of the present invention.

FIG. 1B illustrates a side perspective view of a feeding tube aperture, in accordance with one embodiment of the present invention.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention. However, the order of description should not be construed as to imply that these operations are necessarily order dependent. In particular, these operations need not be performed in the order of presentation.

The phrase “in one embodiment” is utilized repeatedly. The phrase generally does not refer to the same embodiment, however, it may. The terms “comprising”, “having” and “including” are synonymous, unless the context dictates otherwise.

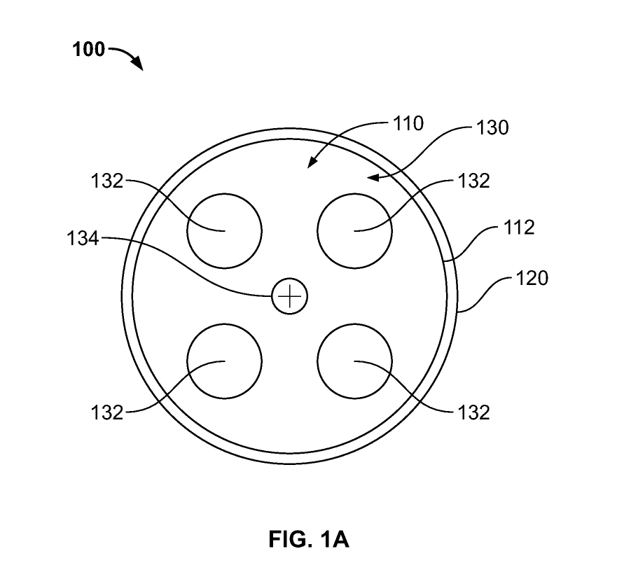

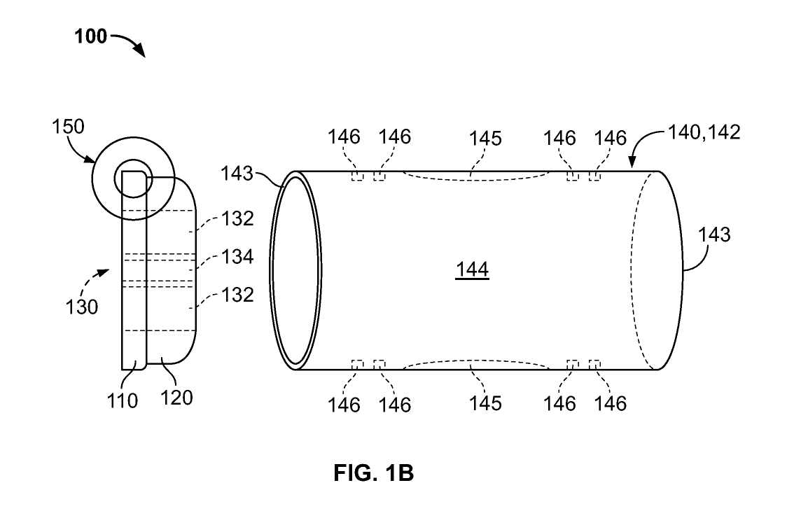

FIG. 1A illustrates a front perspective view of a feeding tube aperture 100, in accordance with one embodiment of the present invention.

The feeding tube aperture 100 includes a generally domed base 110, a flange 120 and a plurality of feeding apertures 130. The feeding tube aperture 100 facilitates any suitable gavage process. The feeding tube aperture 100 secures a feeding tube (not shown) in a perpendicular position in relation to a user's feeding opening (not shown) without tearing into the user's surrounding body tissue, which results in relatively quicker healing and relatively fewer complications. The feeding tube aperture 100 is made of plastic material although the feeding tube aperture 100 can be made of other suitable materials as well. The feeding tube aperture 100 is approximately 0.75 inch in thickness, although the feeding tube aperture 100 can be other suitable thicknesses as well. The generally domed base 110 includes a perimeter 112 and receives the feeding tube and reduces contagions and allergic reactions that might otherwise occur during the gavage process. The flange 120 is disposed below the perimeter 112 of the generally domed base 110 and provides additional support to the feeding tube aperture 100. The flange 120 is approximately 2.00 inches in diameter although the flange 120 can be other suitable diameters as well.

The feeding apertures 130 are disposed on the generally domed base 110 and extend through the feeding tube aperture 100. As illustrated in FIG. 1A, there are 4 relatively large apertures 132 disposed around 1 relatively small aperture 134. The relatively large apertures 132 are uniformly spaced and disposed around the relatively small aperture 134, which is centered on the generally domed base 120. The feeding tube aperture 100 can have other suitable orientations as well regarding the relatively large apertures 132 and the relatively small aperture 134 however the previously described orientation between the relatively large apertures 132 and the relatively small aperture 134 provides a relatively strong flow of food to the user from the gavage process. Other suitable orientations between the relatively large apertures 132 and the relatively small aperture 134 can also be incorporated on the generally domed base 110. The relatively large apertures 132 are approximately 0.50 inch in diameter and the relatively small aperture 134 is approximately 0.25 inch in diameter although the relatively large apertures 132 and the relatively small aperture 134 can be other suitable diameters as well.

FIG. 1B illustrates a side perspective view of a feeding tube aperture 100, in accordance with one embodiment of the present invention.

The feeding tube aperture 100 utilizes all of the features and components previously described in FIG. 1A and its description. The feeding tube aperture 100 also includes a plastic tube anchor 140 and one or more foam booties 150. The plastic tube anchor 140 has a generally cylindrical shape 142 and secures the feeding tube aperture 100 in place within a feeding opening disposed on a user's body. The plastic tube anchor 140 has a raised perimeter edge 143 to enhance safety to secure the feeding tube aperture 100. The plastic tube anchor 140 can also be crimped 145 on the interior 144 of the plastic tube anchor 140 to prevent the plastic tube anchor 140 from sliding or slipping. The plastic tube anchor 140 can also have a plurality of internal raised nubs 146 disposed on the interior 144 of the plastic tube anchor 140 to prevent the plastic tube anchor 140 from sliding or slipping. The foam booties 150 are placed between the feeding tube aperture 100 and the user's feeding opening and serve as feet to comfortably secure the feeding tube aperture 100 within the user's feeding opening.

The feeding tube aperture is a clever way to make the gavage nourishing process relatively quick and effortless. The feeding tube aperture incorporates a durable plastic tube aperture for optimum support and reassurance. The design of the feeding tube aperture includes a sterilized lining with a smooth finish to reduce possible contagions and allergic reactions. The feeding tube aperture will benefit consumers who struggle with painful, scratchy, conventional feeding tube openings and may be readily available in medical retail stores. The feeding tube aperture is the only product of its kind that when placed through the skin it does not tear or cause discomfort of the patient's internal tissue linings or organs. The design of the feeding tube aperture is suitable for all gastroenterology patients and for use in medical offices and hospitals worldwide.

While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention.

1. A feeding tube aperture utilized during a gavage process, comprising:
a generally domed base that includes a perimeter and receives said feeding tube and reduces one or more contagions and allergic reactions that might occur during said gavage process;
a flange that is disposed below said perimeter of said generally domed base and provides additional support to said feeding tube aperture;
a plurality of feeding apertures that are disposed on said generally domed base and extend through said feeding tube aperture;
a generally cylindrical shaped plastic tube anchor with an interior that secures said feeding tube aperture in place within a feeding opening disposed on a user's body; and
one or more foam booties that are placed between said feeding tube aperture and said feeding opening to comfortably secure said feeding tube aperture within said feeding opening.

2. The aperture according to claim 1, wherein said feeding tube apertures include 4 large apertures and one small aperture.

3. The aperture according to claim 2, wherein said apertures are uniformly spaced.

4. The aperture according to claim 2, wherein said small aperture is centered on said generally domed base.

5. The aperture according to claim 1, wherein said plastic tube anchor has a raised perimeter edge to enhance safety to secure said feeding tube aperture.

6. The aperture according to claim 1, wherein said plastic tube anchor is crimped on said interior of said plastic tube anchor to prevent said plastic tube anchor from sliding or slipping.

7. The aperture according to claim 1, wherein said feeding tube aperture secures a feeding tube in a perpendicular position in relation to said feeding opening.

8. A plastic feeding tube aperture utilized during a gavage process, comprising:
a generally domed base that includes a perimeter and receives said feeding tube and reduces one or more contagions and allergic reactions that might occur during said gavage process;
a flange that is disposed below said perimeter of said generally domed base and provides additional support to said feeding tube aperture;
a plurality of feeding apertures that include 4 large apertures and one small aperture that are disposed on said generally domed base and extend through said feeding tube aperture;
a generally cylindrical shaped plastic tube anchor with an interior that secures said feeding tube aperture in place within a feeding opening disposed on a user's body that has a raised perimeter edge to enhance safety to secure said feeding tube aperture; and
one or more foam booties that are placed between said feeding tube aperture and said feeding opening to comfortably secure said feeding tube aperture within said feeding opening.

9. The aperture according to claim 9, wherein said apertures are uniformly spaced.

10. The aperture according to claim 8, wherein said small aperture is centered on said generally domed base.

11. The aperture according to claim 8, wherein said plastic tube anchor is crimped on said interior of said plastic tube anchor to prevent said plastic tube anchor from sliding or slipping.

12. The aperture according to claim 8, wherein said plastic tube anchor has a plurality of internal raised nubs disposed on said interior of said plastic tube anchor to prevent said plastic tube anchor from sliding or slipping.

13. The aperture according to claim 8, wherein said feeding tube aperture secures a feeding tube in a perpendicular position in relation to said feeding opening.

14. A plastic feeding tube aperture utilized during a gavage process, comprising:
a generally domed base that includes a perimeter and receives said feeding tube and reduces one or more contagions and allergic reactions that might occur during said gavage process;
a flange that is disposed below said perimeter of said generally domed base and provides additional support to said feeding tube aperture;
a plurality of uniformly spaced feeding apertures that include 4 large apertures and one small aperture that are disposed on said generally domed base and extend through said feeding tube aperture;
a generally cylindrical shaped plastic tube anchor with an interior that secures said feeding tube aperture in place within a feeding opening disposed on a user's body that has a raised perimeter edge to enhance safety to secure said feeding tube aperture; and
one or more foam booties that are placed between said feeding tube aperture and said feeding opening to comfortably secure said feeding tube aperture within said feeding opening.

15. The aperture according to claim 14, wherein said small aperture is centered on said generally domed base.

16. The aperture according to claim 14, wherein said plastic tube anchor is crimped on said interior of said plastic tube anchor to prevent said plastic tube anchor from sliding or slipping.

17. The aperture according to claim 14, wherein said plastic tube anchor has a plurality of internal raised nubs disposed on said interior of said plastic tube anchor to prevent said plastic tube anchor from sliding or slipping.

18. The aperture according to claim 14, wherein said feeding tube aperture secures a feeding tube in a perpendicular position in relation to said feeding opening.

19. The aperture according to claim 14, wherein said large apertures are approximately 0.50 inch in diameter.

20. The aperture according to claim 14, wherein said small aperture is approximately 0.25 inch in diameter.
